(19) United States
(12) Patent Application Publication
(10) Pub. No.: US 2025/0275741 A1
(43) Pub. Date: Sep. 4, 2025

(54) Medical X-Ray Imaging Device and Power Supply System Therefor

(71) Applicant: Siemens Shanghai Medical Equipment Ltd., Shanghai (CN)

(72) Inventor: Zhida Chen, Shanghai (CN)

(73) Assignee: Siemens Shanghai Medical Equipment Ltd., Shanghai (CN)

(21) Appl. No.: 18/863,692

(22) PCT Filed: Apr. 20, 2023
(86) PCT No.: PCT/CN2023/089571

§ 371 Date: Nov. 7, 2024

(30) Foreign Application Priority Data
May 10, 2022 (CN) 202221198333.6

(51) Int. Cl. A61B 6/00; H02J 9/06; H05G 1/10

(52) U.S. Cl. CPC A61B 6/56; H02J 9/061; H02J 9/062; H05G 1/10

(57) ABSTRACT

A power supply system for a medical X-ray imaging device is described, which includes power connector configured to be connected to a power grid. The power supply system can efficiently reduce the time for reconnecting the medical X-ray imaging device to a power supply to recover to a normal working state. In addition, the medical X-ray imaging device is also provided.

### CROSS-REFERENCE TO RELATED APPLICATIONS

The present application is a U.S. national stage entry of PCT application no. PCT/CN2023/089571, filed Apr. 20, 2023, which claims priority to and the benefit of China patent application no. CN 202221198333.6, filed on May 10, 2022, the contents of each of which are incorporated herein by reference in their entireties.

### TECHNICAL FIELD

The present disclosure relates to a power supply system and, in particular, to a power supply system for a medical X-ray imaging device and the medical X-ray imaging device including the power supply system.

### BACKGROUND

At present, movable products of a medical X-ray imaging device, such as a movable C-shaped arm X-ray machine, have been developed. The movable products can easily move between different operating rooms of a hospital. In this process, it is necessary to disconnect and reconnect a power supply as well as close and restart the device. This process takes a long time, and the device cannot be used in an emergency.

### SUMMARY

One objective of the present disclosure is to provide a power supply system for a medical X-ray imaging device. The power supply system can reduce the time for reconnecting the medical X-ray imaging device to a power supply to recover to a normal working state.

Another objective of the present disclosure is to provide the medical X-ray imaging device. The power supply system for the medical X-ray imaging device can reduce the time for reconnecting the medical X-ray imaging device to the power supply to recover to the normal working state.

The present disclosure provides a power supply system for a medical X-ray imaging device. The power supply system includes a power connector, a first power supply connector, a battery module, and a second power supply connector. The power connector is configured to be connected to a power grid. The first power supply connector is connected to the power connector. The first power supply connector is configured to be connected to a first-class electrical component of the medical X-ray imaging device. The battery module is connected to the power connector. The battery module is provided with a storage battery. The battery module is configured to acquire electrical energy from the power grid through the power connector to charge the storage battery. The second power supply connector is connected to the battery module. The second power supply connector is configured to be connected to a second-class electrical component of the medical X-ray imaging device. The battery module is configured to connect any one of the power connector and the storage battery to the second power supply connector in a switchable mode.

According to the power supply system for a medical X-ray imaging device, the second-class electrical component can obtain electrical energy from the storage battery to maintain operating in a case that the power connector is in a power-off state, so that when the power connector is reconnected to the power grid, the second-class electrical component does not need to be restarted, and only the electrical component with the restarting time less than a preset time threshold, e.g. the first-class electrical component, needs to be restarted. Therefore, the power supply system can effectively reduce the time for reconnecting the medical X-ray imaging device to the power supply to recover to the normal working state.

In another exemplary embodiment of the power supply system for a medical X-ray imaging device, the battery module includes a first switch. The first switch is controlled by the power connector. A contact end of the first switch is connected to the power connector, the storage battery, and the second power supply connector. The first switch is configured to connect the power connector to the second power supply connector in a case that the power connector is in a power-on state; and the first switch is configured to connect the storage battery to the second power supply connector in a case that the power connector is in a power-off state. By arranging the first switch, connection of the power connector, the storage battery, and the second power supply connector can be automatically switched according to a condition whether the power connector is powered on or not, and therefore the convenience in use can be improved.

In yet another exemplary embodiment of the power supply system for a medical X-ray imaging device, the first switch is a relay.

In still yet another exemplary embodiment of the power supply system for a medical X-ray imaging device, the battery module includes a first AC/DC converter and a first DC/AC converter. The first AC/DC converter is connected between the power connector and the storage battery. The first DC/AC converter is connected between the storage battery and the first switch. Therefore, the electrical energy can be switched between alternating current and direct current according to the requirements of the storage battery and AC-powered electrical components.

In still yet another exemplary embodiment of the power supply system for a medical X-ray imaging device, the power supply system further includes a second AC/DC converter. The second AC/DC converter is connected to the first power supply connector and is configured to be connected to a DC-powered first-class electrical component. Therefore, the electrical energy is conveniently supplied to the DC-powered first-class electrical component.

In still yet another exemplary embodiment of the power supply system for a medical X-ray imaging device, the power supply system further includes a third AC/DC converter. The third AC/DC converter is connected to the second power supply connector and is configured to be connected to a DC-powered second-class electrical component. Therefore, the electrical energy is conveniently supplied to the DC-powered second-class electrical component.

In still yet another exemplary embodiment of the power supply system for a medical X-ray imaging device, the power supply system includes two second power supply connectors; one second power supply connector is configured to be connected to an AC-powered second-class electrical component, and the other second power supply connector is configured to be connected to the DC-powered second-class electrical component. The contact end of the first switch is provided with a first contact set and a second contact set. The first contact set is connected to the second power supply connector which is configured to be connected to the AC-powered second-class electrical component, and the second contact set is connected to the second power supply connector which is configured to be connected to the DC-powered second-class electrical component. The battery module further includes a first AC/DC converter, a first DC/AC converter and a DC/DC converter. The first AC/DC converter is connected between the power connector and the storage battery. The first DC/AC converter is connected between the storage battery and the first contact set. The DC/DC converter is connected between the storage battery and the second contact set. Therefore, the electrical energy can be switched between alternating current and direct current according to the requirements of the storage battery and the electrical components.

In still yet another exemplary embodiment of the power supply system for a medical X-ray imaging device, the power supply system further includes a second AC/DC converter. The second AC/DC converter is connected to the first power supply connector and configured to be connected to the DC-powered first-class electrical component. The second contact set is connected to the power connector through the second AC/DC converter and the first power supply connector. Therefore, a circuit can be simplified, and the stability of the power supply system can be improved.

In still yet another exemplary embodiment of the power supply system for a medical X-ray imaging device, the battery module further includes a battery management system. The battery management system is configured to detect a residual electric quantity of the storage battery and transmit an electric quantity signal. The power supply system further includes a prompt module. The prompt module is connected to the battery management system and is configured to display patterns or make a sound according to the electric quantity signal. Therefore, a user can conveniently know the residual electric quantity of the storage battery.

In still yet another exemplary embodiment of the power supply system for a medical X-ray imaging device, the battery module is an uninterruptible power supply. The power supply system further includes a second switch and a third power supply connector. The third power supply connector is configured to be connected to a second-class electrical component needing limitation on operating power in power off. The second switch is controlled by the power connector. A contact end of the second switch is connected to the power connector, the second power supply connectors and the third power supply connector. The second switch is configured to connect the power connector to the third power supply connector in a case that the power connector is in the power-on state, and the second switch is configured to connect the second power supply connectors to the third power supply connector in a case that the power connector is in the power-off state. By arranging the uninterruptible power supply, the circuit can be simplified, and the stability of the power supply system can be improved. By arranging the second switch and the third power supply connector, a situation that the maximum output power of the uninterruptible power supply limits the operating power of the electrical components in a case that the power connector is in the power-on state can be avoided.

In still yet another exemplary embodiment of the power supply system for a medical X-ray imaging device, the second switch is a relay.

In still yet another exemplary embodiment of the power supply system for a medical X-ray imaging device, the power supply system further includes a monitoring module. The monitoring module is configured to detect an on-off state of the contact end of the second switch and transmit an operating power limiting signal in a case of detecting that the second switch connects the second power supply connectors to the third power supply connector. A control system for a medical X-ray imaging device is configured to control the second-class electrical component needing limitation on operating power in power off to only implement a function that the power is less than a set threshold according to the operating power limiting signal. Therefore, a situation that the device is damaged because of forcibly operating a function that the power is greater than the maximum output power of the uninterruptible power supply in a case that the power connector is in the power-off state can be prevented.

In still yet another exemplary embodiment of the power supply system for a medical X-ray imaging device, the power supply system further includes an AC/AC converter. The AC/AC converter is connected between the power connector and the first power supply connector and between the power connector and the battery module.

The present disclosure further provides a medical X-ray imaging device. The medical X-ray imaging device includes the abovementioned power supply system. The power supply system for the medical X-ray imaging device can effectively reduce the time for reconnecting the medical X-ray imaging device to the power supply to recover to the normal working state.

In another exemplary embodiment of the medical X-ray imaging device, the electrical components of the medical X-ray imaging device are divided into the first-class electrical component and the second-class electrical component according to the time for restarting; the restarting time of the first-class electrical component is less than a preset time threshold; and the restarting time of the second-class electrical component is greater than or equal to the preset time threshold.

In yet another exemplary embodiment of the medical X-ray imaging device, the medical X-ray imaging device is a movable C-shaped arm X-ray machine; and the second-class electrical component of the movable C-shaped arm X-ray machine includes an X-ray generator, a computer, a collimator and a micro-control unit.

### BRIEF DESCRIPTION OF THE DRAWINGS

The following drawings are only used to schematically explain the present disclosure, and do not limit the scope of the present disclosure.

FIG. 1 illustrates a schematic circuit diagram of an example power supply system for a medical X-ray imaging device, in accordance with an embodiment;

FIG. 2 illustrates a schematic circuit diagram of an example power supply system for a medical X-ray imaging device, in accordance with an embodiment; and

FIG. 3 illustrates a schematic circuit diagram of an example power supply system for a medical X-ray imaging device, in accordance with an embodiment.

### REFERENCE NUMERALS

10, power connector
21, first power supply connector
22, second power supply connector
23, third power supply connector
30, battery module
31, storage battery
32, first switch
33, first AC/DC converter
34, first DC/AC converter
35, DC/DC converter
36, battery management system
42, second AC/DC converter
43, third AC/DC converter
44, AC/AC converter
50, prompt module
60, second switch
70, monitoring module
81, AC-powered first-class electrical component
82, DC-powered first-class electrical component
91, AC-powered second-class electrical component
911, AC-powered second-class electrical component needing limitation on operating power in power off
912, AC-powered second-class electrical component not needing limitation on operating power in power off
92, DC-powered second-class electrical component

### DETAILED DESCRIPTION OF THE DISCLOSURE

In order to have a clearer understanding of the technical characteristics, objectives, and effects of the present disclosure, the specific embodiments of the present disclosure are explained with reference to the accompanying drawings. The same reference numerals in each figure represent components with the same structure or similar structure and the same function.

The present disclosure provides a power supply system for a medical X-ray imaging device. Electrical components of the medical X-ray imaging device are divided into a first-class electrical component and a second-class electrical component according to the time for restarting; the restarting time of the first-class electrical component is less than a preset time threshold; and the restarting time of the second-class electrical component is greater than or equal to the preset time threshold. The specific numerical value of the time threshold can be flexibly adjusted according to actual needs. The medical X-ray imaging device can be, for example, a movable C-shaped arm X-ray machine. The second-class electrical component of the movable C-shaped arm X-ray machine includes an X-ray generator, a computer, a collimator and a micro-control unit.

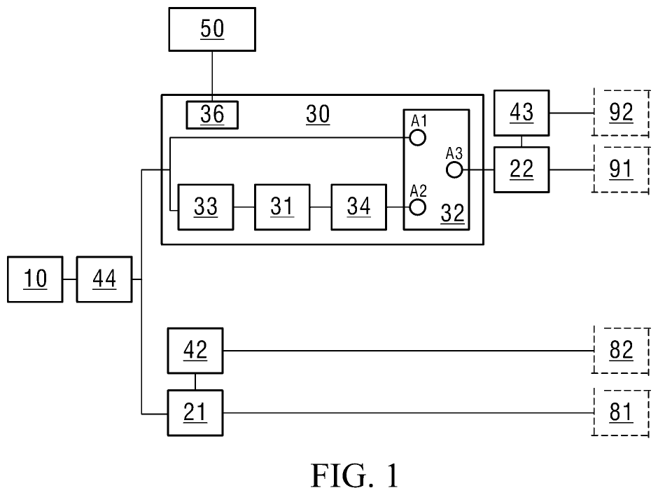

FIG. 1 illustrates a schematic circuit diagram of an example power supply system for a medical X-ray imaging device, in accordance with an embodiment. As shown in FIG. 1, the power supply system for a medical X-ray imaging device includes a power connector 10, a first power supply connector 21, a battery module 30, and a second power supply connector 22.

The power connector 10 is configured to be connected to a power grid. The first power supply connector 21 is connected to the power connector 10. The first power supply connector 21 is configured to be connected to a first-class electrical component (including an AC-powered first-class electrical component 81 and a DC-powered first-class electrical component 82) of the medical X-ray imaging device. The battery module 30 is connected to the power connector 10. The battery module 30 is provided with a storage battery 31. The battery module 30 is configured to acquire electrical energy from the power grid through the power connector 10 to charge the storage battery 31. The second power supply connector 22 is connected to the battery module 30. The second power supply connector 22 is configured to be connected to a second-class electrical component (including an AC-powered second-class electrical component 91 and a DC-powered second-class electrical component 92) of the medical X-ray imaging device. The battery module 30 is configured to connect any one of the power connector 10 and the storage battery 31 to the second power supply connector 22 in a switchable mode. For example, switching is achieved in a manual control or automatic control mode. In this exemplary embodiment, the battery module 30 achieves automatic switching through a switch, but it is not limited to this.

The working process of the power supply system is illustrated as follows.

In a case that the power connector 10 is in a power-on state (e.g., the power connector 10 is connected to the power grid), the first-class electrical component (including the AC-powered first-class electrical component 81 and the DC-powered first-class electrical component 82) can obtain electrical energy from the power grid through the power connector 10. Meanwhile, the battery module 30 is switched to connect the power connector 10 to the second power supply connector 22, and the second-class electrical component (including the AC-powered second-class electrical component 91 and the DC-powered second-class electrical component 92) can obtain electrical energy from the power grid through the power connector 10. Meanwhile, the battery module 30 can obtain the electrical energy from the power grid through the power connector 10 to charge the storage battery 31.

In a case that the power connector 10 is in a power-off state (e.g., the power connector 10 is not connected to the power grid), the first-class electrical component (including the AC-powered first-class electrical component 81 and the DC-powered first-class electrical component 82) is powered off. Meanwhile, the battery module 30 is switched to connect the storage battery 31 to the second power supply connector 22, and the second-class electrical component (including the AC-powered second-class electrical component 91 and the DC-powered second-class electrical component 92) can obtain electrical energy from the storage battery 31 to maintain operating.

When the power connector 10 is reconnected to the power grid, the first-class electrical component (including the AC-powered first-class electrical component 81 and the DC-powered first-class electrical component 82) can obtain electrical energy from the power grid through the power connector 10 to be restarted. Meanwhile, the battery module 30 is switched to connect the power connector 10 to the second power supply connector 22, and the second-class electrical component (including the AC-powered second-class electrical component 91 and the DC-powered second-class electrical component 92) can obtain electrical energy from the power grid through the power connector 10. Meanwhile, the battery module 30 can obtain electrical energy from the power grid through the power connector 10 to charge the storage battery 31 again.

As the second-class electrical component can obtain electrical energy from the storage battery 31 to maintain operating in a case that the power connector 10 is in the power-off state, when the power connector 10 is reconnected to the power grid, the second-class electrical component does not need to be restarted, and only the electrical component with the restarting time less than a preset time threshold, e.g. the first-class electrical component, needs to be restarted. Therefore, the power supply system can effectively reduce the time for reconnecting the medical X-ray imaging device to the power supply to recover to the normal working state.

As shown in FIG. 1, in this exemplary embodiment, the battery module 30 includes a first switch 32 (only a contact end of the first switch is drawn in FIG. 1). The first switch 32 is controlled by the power connector 10, for example, the first switch is a relay, but it is not limited to this. The contact end of the first switch 32 is connected to the power connector 10, the storage battery 31 and the second power supply connector 22, specifically, a contact A1 is connected to the power connector 10, a contact A2 is connected to the storage battery 31, and a contact A3 is connected to the second power supply connector 22. The first switch 32 is configured to connect the power connector 10 to the second power supply connector 22 in a case that the power connector 10 is in a power-on state (e.g. the contact A1 makes contact with the contact A3, and the contact A2 is disconnected from the contact A3), and the first switch 32 is configured to connect the storage battery 31 to the second power supply connector 22 in a case that the power connector 10 is in a power-off state (e.g., the contact A2 makes contact with the contact A3, and the contact A1 is disconnected from the contact A3). By arranging the first switch, connection of the power connector 10, the storage battery 31, and the second power supply connector 22 can be automatically switched according to a condition whether the power connector 10 is powered on or not, and therefore the convenience in use can be improved.

As shown in FIG. 1, in this exemplary embodiment, the battery module 30 further includes a first AC/DC converter 33 and a first DC/AC converter 34. The first AC/DC converter 33 is connected between the power connector 10 and the storage battery 31. The first DC/AC converter 34 is connected between the storage battery 31 and the contact A2 of the first switch 32. As current in the power grid is alternating current, the storage battery 31 is charged with direct current, and current in some electrical components is alternating current; and the first AC/DC converter 33 and the first DC/AC converter 34 are arranged to switch electrical energy between alternating current and direct current according to the requirements of the storage battery 31 and AC-powered electrical components.

As shown in FIG. 1, in this exemplary embodiment, the power supply system further includes a second AC/DC converter 42 and a third AC/DC converter 43. The second AC/DC converter 42 is connected to the first power supply connector 21 and configured to be connected to the DC-powered first-class electrical component 82. The AC-powered first-class electrical component 81 is directly connected to the first power supply connector 21. The third AC/DC converter 43 is connected to the second power supply connector 22 and configured to be connected to the DC-powered second-class electrical component 92. The AC-powered second-class electrical component 91 is directly connected to the second power supply connector 22. Electrical energy can be conveniently supplied to the DC-powered electrical components through the second AC/DC converter 42 and the third AC/DC converter 43.

As shown in FIG. 1, in this exemplary embodiment, the power supply system further includes an AC/AC converter 44. The AC/AC converter 44 is connected between the power connector 10 and the first power supply connector 21 and between the power connector 10 and the battery module 30. Therefore, the alternating current provided by the power grid is converted into alternating current meeting the requirements of the medical X-ray imaging device.

As shown in FIG. 1, in this exemplary embodiment, the battery module 30 further includes a battery management system 36. The battery management system 36 is configured to detect a residual electric quantity of the storage battery 31 and transmit an electric quantity signal. The power supply system further includes a prompt module 50. The prompt module 50 is connected to the battery management system 36 and is configured to display patterns or make a sound according to the electric quantity signal. The prompt module 50 may comprise a display for example, and is configured to display different patterns to represent different residual electric quantities of the storage battery 31. The prompt module 50 may also comprise a buzzer for example, and is configured to make a sound to remind a user when the residual electric quantity of the storage battery 31 is less than a threshold.

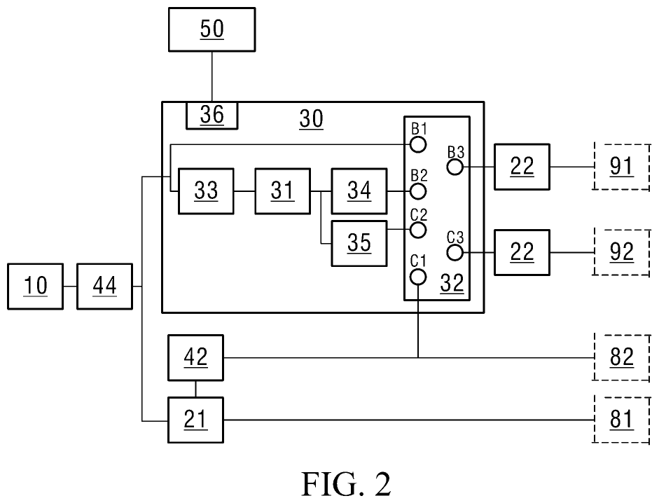

FIG. 2 illustrates a schematic circuit diagram of an example power supply system for a medical X-ray imaging device, in accordance with an embodiment. As shown in FIG. 2, the power supply system for a medical X-ray imaging device includes a power connector 10, a first power supply connector 21, a battery module 30, and two second power supply connectors 22.

The power connector 10 is configured to be connected to a power grid. The first power supply connector 21 is connected to the power connector 10. The first power supply connector 21 is configured to be connected to a first-class electrical component (including an AC-powered first-class electrical component 81 and a DC-powered first-class electrical component 82) of the medical X-ray imaging device. The battery module 30 is connected to the power connector 10. The battery module 30 is provided with a storage battery 31. The battery module 30 is configured to acquire electrical energy from the power grid through the power connector 10 to charge the storage battery 31. The second power supply connector 22 is connected to the battery module 30. The second power supply connectors 22 are configured to be connected to a second-class electrical component of the medical X-ray imaging device; one second power supply connector 22 is configured to be connected to an AC-powered second-class electrical component 91, and the other second power supply connector 22 is configured to be connected to a DC-powered second-class electrical component 92. The battery module 30 is configured to connect any one of the power connector 10 and the storage battery 31 to the second power supply connectors 22 in a switchable mode.

Specifically, as shown in FIG. 2, the battery module 30 includes a first switch 32 (only a contact end of the first switch is drawn in FIG. 2). The first switch 32 is controlled by the power connector 10, for example, the first switch is a relay, but it is not limited to this. The contact end of the first switch 32 is provided with a first contact set (i.e., contacts B1, B2 and B3) and a second contact set (i.e., contacts C1, C2 and C3). In the first contact set, the contact B1 is connected to the power connector 10, the contact B2 is connected to the storage battery 31, and the contact B3 is connected to the second power supply connector 22 which is configured to be connected to the AC-powered second-class electrical component 91. In the second contact set, the contact C1 is connected to the power connector 10, the contact C2 is connected to the storage battery 31, and the contact C3 is connected to the second power supply connector 22 which is configured to be connected to the DC-powered second-class electrical component 92.

As shown in FIG. 2, in this exemplary embodiment, the battery module 30 further includes a first AC/DC converter 33, a first DC/AC converter 34, and a DC/DC converter 35. The first AC/DC converter 33 is connected between the power connector 10 and the storage battery 31. The first DC/AC converter 34 is connected between the storage battery 31 and the contact B2 of the first contact set. The DC/DC converter 35 is connected between the storage battery 31 and the contact C2 of the second contact set.

The first switch 32 is configured to connect the power connector 10 to the second power supply connectors 22 (namely, the contact B1 makes contact with the contact B3, and the contact C1 makes contact with the contact C3) in a case that the power connector 10 is in a power-on state; and the first switch 32 is configured to connect the storage battery 31 to the second power supply connectors 22 (namely, the contact B2 makes contact with the contact B3, and the contact C2 makes contact with the contact C3) in a case that the power connector 10 is in a power-off state. By arranging the first switch, connection of the power connector 10, the storage battery 31, and the second power supply connector 22 can be automatically switched according to a condition whether the power connector 10 is powered on or not, and therefore the convenience in use can be improved.

As shown in FIG. 2, in this exemplary embodiment, the power supply system further includes a second AC/DC converter 42. The second AC/DC converter 42 is connected to the first power supply connector 21 and configured to be connected to the DC-powered first-class electrical component 82. The AC-powered first-class electrical component 81 is directly connected to the first power supply connector 21. The contact C1 of the second contact set is connected to the power connector 10 through the second AC/DC converter 42 and the first power supply connector 21. Electrical energy can be conveniently supplied to the DC-powered electrical components through the second AC/DC converter 42.

As shown in FIG. 2, in this exemplary embodiment, the power supply system further includes an AC/AC converter 44. The AC/AC converter 44 is connected between the power connector 10 and the first power supply connector 21 and between the power connector 10 and the battery module 30. Therefore, the alternating current provided by the power grid is converted into alternating current meeting the requirements of the medical X-ray imaging device.

As shown in FIG. 2, in this exemplary embodiment, the battery module 30 further includes a battery management system 36. The battery management system 36 is configured to detect a residual electric quantity of the storage battery 31 and transmit an electric quantity signal. The power supply system further includes a prompt module 50. The prompt module 50 is connected to the battery management system 36 and is configured to display patterns or make a sound according to the electric quantity signal. The prompt module 50 is a display for example, and is configured to display different patterns to represent different residual electric quantities of the storage battery 31. The prompt module 50 is also a buzzer for example, and is configured to make a sound to remind a user when the residual electric quantity of the storage battery 31 is less than a threshold.

The schematic explanation of the working process of the power supply system is the same as that of the working process of the power supply system shown in FIG. 1, so it is not repeated herein.

As the second-class electrical component can obtain electrical energy from the storage battery 31 to maintain operating in a case that the power connector 10 is in the power-off state, when the power connector 10 is reconnected to the power grid, the second-class electrical component does not need to be restarted, and only the electrical component with the restarting time less than a preset time threshold, namely the first-class electrical component, needs to be restarted. Therefore, the power supply system can effectively reduce the time for reconnecting the medical X-ray imaging device to the power supply to recover to the normal working state.

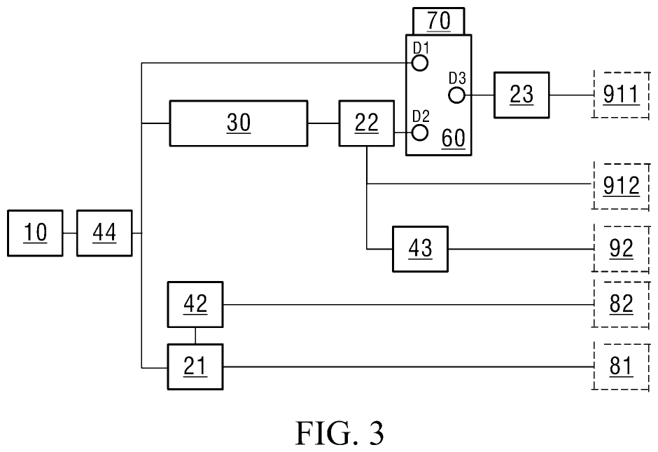

FIG. 3 illustrates a schematic circuit diagram of an example power supply system for a medical X-ray imaging device, in accordance with an embodiment. The power supply system shown in FIG. 3 may be the same as or similar to the power supply system shown in FIG. 1, and is not repeated herein, with the differences being described as follows.

As shown in FIG. 3, in this exemplary embodiment, the battery module 30 is an uninterruptible power supply. A second-class electrical component of the medical X-ray imaging device can be divided into a second-class electrical component needing limitation on operating power in power off and a second-class electrical component not needing limitation on operating power in power off according to, for example, the maximum operating power in operation. The maximum operating power of the second-class electrical component needing limitation on operating power in power off is greater than the maximum output power of the uninterruptible power supply, for example, the second-class electrical component is an X-ray generator, the exposure power of the second-class electrical component is greater than the maximum output power of the uninterruptible power supply, and the power for maintaining the power-on state is less than or equal to the maximum output power of the uninterruptible power supply. The maximum operating power of the second-class electrical component not needing limitation on operating power in power off is less than or equal to the maximum output power of the uninterruptible power supply.

As shown in FIG. 3, in this exemplary embodiment, the power supply system further includes a second switch 60 (only a contact end of the second switch is drawn in FIG. 3) and a third power supply connector 23. The third power supply connector 23 is configured to be connected to an AC-powered second-class electrical component 911 needing limitation on operating power in power off in second-class electrical component. An AC-powered second-class electrical component 912 not needing limitation on operating power in power off is directly connected to the second power supply connectors 22. The second switch 60 is controlled by the power connector 10, for example, the second switch is a relay, but it is not limited to this. In the contact end of the second switch 60, a contact D1 is connected to the power connector 10, a contact D2 is connected to the second power supply connectors 22, and a contact D3 is connected to the third power supply connector 23. The second switch 60 is configured to connect the power connector 10 to the third power supply connector 23 in a case that the power connector 10 is in the power-on state (namely the contact D1 makes contact with the contact D3). The second switch 60 is configured to connect the second power supply connectors 22 to the third power supply connector 23 in a case that the power connector 10 is in the power-off state (namely the contact D2 makes contact with the contact D3). In a case that the second power supply connector 22 is connected to the third power supply connector 23, the AC-powered second-class electrical component 911 needing limitation on operating power can only start a function that the operating power is less than or equal to the maximum output power of the uninterruptible power supply. For example, the X-ray generator can only maintain the power-on state and cannot be exposed. By arranging the uninterruptible power supply, the circuit can be simplified, and the stability of the power supply system can be improved. By arranging the second switch 60 and the third power supply connector 23, a situation that the maximum output power of the uninterruptible power supply limits the operating power of the electrical components in a case that the power connector 10 is in the power-on state can be avoided.

As shown in FIG. 3, in this exemplary embodiment, the power supply system further includes a monitoring module 70. The monitoring module 70 is configured to detect an on-off state of the contact end of the second switch 60 and transmit an operating power limiting signal in a case of detecting that the second switch 60 connects the second power supply connectors 22 to the third power supply connector 23. A control system for a medical X-ray imaging device is configured to control the second-class electrical component needing limitation on operating power in power off to only implement a function that the power is less than a set threshold (the maximum output power of the uninterruptible power supply is not exceeded) according to the operating power limiting signal. For example, the X-ray generator can only maintain the power-on state and cannot be exposed. Therefore, a situation that the device is damaged because of forcibly operating a function that the power is greater than the maximum output power of the uninterruptible power supply in a case that the power connector 10 is in the power-off state can be prevented.

The present disclosure further provides a medical X-ray imaging device. In one exemplary embodiment, the medical X-ray imaging device includes any one of the abovementioned power supply systems. The electrical components of the medical X-ray imaging device are divided into the first-class electrical component and the second-class electrical component according to the time for restarting; the restarting time of the first-class electrical component is less than a preset time threshold; and the restarting time of the second-class electrical component is greater than or equal to the preset time threshold. The power supply system for the medical X-ray imaging device can effectively reduce the time for reconnecting the medical X-ray imaging device to the power supply to recover to the normal working state.

In this exemplary embodiment, the medical X-ray imaging device is a movable C-shaped arm X-ray machine, and second-class electrical component of the movable C-shaped arm X-ray machine includes an X-ray generator, a computer, a collimator and a micro-control unit; and the X-ray generator belongs to the second-class electrical component needing limitation on operating power in power off.

It is to be understood that, although this description is described according to each embodiment, each embodiment may not include only one independent technical solution. The narrative mode of this description is merely for clarity. This description should be considered as a whole by a person skilled in the art, and the technical solution in each embodiment may also be properly combined, to form other embodiments that can be understood by a person skilled in the art.

The series of detailed explanations listed above are only specific explanations for the feasible embodiments of the present disclosure, and are not intended to limit the scope of protection of the present disclosure. Any equivalent implementations or changes without departing from the technical spirit of the present disclosure, such as the combination, segmentation, or repetition of features, should be included in the scope of protection of the present disclosure.

In the present disclosure, “exemplary” means “serving as an instance, example, or explanation”, and any illustration or embodiment described as “exemplary” in the present disclosure cannot be interpreted as a more preferred or advantageous technical solution.

In the present disclosure, “first”, “second”, etc. are not used to indicate their importance or order, but only to indicate their differences and facilitate the description of the document.

To make the drawings concise, only the parts related to the present disclosure are schematically represented in each figure, and they do not represent the actual structure of a product.

In the present disclosure, “AC” represents alternating current, and “DC” represents direct current. An AC/DC converter refers to an element that converts AC into DC. A DC/AC converter refers to an element that converts DC into AC. An AC/AC converter refers to an element that converts AC of one form into AC of another form. A DC/DC converter refers to an element that converts DC of one form into DC of another form.

1.-14. (canceled)

15. A power supply system for a medical X-ray imaging device, comprising:
a power connector configured to be connected to a power grid;
a first power supply connector connected to the power connector,
wherein the first power supply connector is configured to be connected to a first-class electrical component of the medical X-ray imaging device;
a battery module connected to the power connector,
wherein the battery module comprises a storage battery and is configured to acquire electrical energy from the power grid via the power connector to charge the storage battery; and
a second power supply connector connected to the battery module,
wherein the second power supply connector is configured to be connected to a second-class electrical component of the medical X-ray imaging device, and
wherein the battery module is configured to connect one or more of the power connector and the storage battery to the second power supply connector in a switchable mode.

16. The power supply system according to claim 15, wherein:
the battery module comprises a first switch that is controlled via the power connector,
a contact end of the first switch is connected to the power connector, the storage battery, and to the second power supply connector,
the first switch is configured to connect the power connector to the second power supply connector for the power connector being in a power-on state, and to connect the storage battery to the second power supply connector for the power connector being in a power-off state.

17. The power supply system according to claim 16, wherein the battery module comprises:
a first AC/DC converter connected between the power connector and the storage battery; and
a first DC/AC converter connected between the storage battery and the first switch.

18. The power supply system according to claim 15, further comprising:
a second AC/DC converter connected to the first power supply connector and configured to be connected to a DC-powered first-class electrical component.

19. The power supply system according to claim 15, further comprising:
a third AC/DC converter connected to the second power supply connector and configured to be connected to a DC-powered second-class electrical component.

20. The power supply system according to claim 16, further comprising:
two second power supply connectors,
wherein one of the two second power supply connectors is configured to be connected to an AC-powered second-class electrical component, and another one of the two second power supply connectors is configured to be connected to a DC-powered second-class electrical component,
wherein the contact end of the first switch is provided with a first contact set and a second contact set, the first contact set being connected to the second power supply connector that is configured to be connected to the AC-powered second-class electrical component, and
wherein the second contact set is connected to the second power supply connector that is configured to be connected to the DC-powered second-class electrical component.

21. The power supply system according to claim 20, wherein the battery module further comprises:
a first AC/DC converter connected between the power connector and the storage battery;
a first DC/AC converter connected between the storage battery and the first contact set; and
a DC/DC converter connected between the storage battery and the second contact set.

22. The power supply system according to claim 21, further comprising:
a second AC/DC converter connected to the first power supply connector and configured to be connected to a DC-powered first-class electrical component,
wherein the second contact set is connected to the power connector via the second AC/DC converter and the first power supply connector.

23. The power supply system according to claim 15, wherein the battery module further comprises a battery management system configured to detect a residual electric quantity of the storage battery and to transmit an electric quantity signal, and further comprising:
a prompt module connected to the battery management system and configured to display patterns or make a sound according to the electric quantity signal.

24. The power supply system according to claim 15, wherein the battery module comprises an uninterruptible power supply, and further comprising:
a second switch; and
a third power supply connector configured to be connected to a second-class electrical component requiring a limitation on operating power in a power off state,
wherein the second switch is controlled via the power connector,
wherein a contact end of the second switch is connected to the power connector, the second power supply connectors, and to the third power supply connector,
wherein the second switch is configured to connect the power connector to the third power supply connector for the power connector being in a power-on state, and
wherein the second switch is configured to connect the second power supply connector to the third power supply connector for the power connector being in a power-off state.

25. The power supply system according to claim 24, further comprising:
a monitoring module configured to detect an on-off state of the contact end of the second switch and to transmit an operating power limiting signal in response to detecting the second switch connecting the second power supply connectors to the third power supply connector; and
a control system configured to control the second-class electrical component to implement a function in which the power is less than a set threshold according to the operating power limiting signal.

26. The power supply system according to claim 15, further comprising:
an AC/AC converter connected between the power connector and the first power supply connector, and between the power connector and the battery module.

27. The power supply system according to claim 15, wherein:
the first-class electrical component and the second-class electrical component of the power supply system are defined according to a time for restarting,
the restarting time of the first-class electrical component is less than a preset time threshold, and
the restarting time of the second-class electrical component is greater than or equal to the preset time threshold.

28. The power supply system according to claim 15, wherein the medical X-ray imaging device comprises a movable C-shaped arm X-ray machine.

29. The power supply system according to claim 15, wherein the second-class electrical component comprises one of an X-ray generator, a computer, a collimator, or a micro-control unit.
